(19) United States
(12) Patent Application Publication
(10) Pub. No.: US 2021/0322991 A1
(43) Pub. Date: Oct. 21, 2021

(54) RAPID THERMAL CYCLING

(71) Applicant: Hewlett-Packard Development Company, L.P., Spring, TX (US)

(72) Inventors: Diane HAMMERSTAD, Corvallis, OR (US); Alexander GOVYADINOV, Corvallis, OR (US); Brian J. KEEFE, San Diego, CA (US); Erik D. TORNIAINEN, Corvallis, OR (US); Tyler COCHELL, Corvallis, OR (US); Pavel KORNILOVICH, Corvallis, OR (US)

(73) Assignee: Hewlett-Packard Development Company, L.P., Spring, TX (US)

(21) Appl. No.: 17/268,971

(22) PCT Filed: Dec. 13, 2018
(86) PCT No.: PCT/US2018/065498

§ 371 Date: Feb. 17, 2021

(51) Int. Cl. B01L 7/00; B01L 3/00; B01L 9/00

(52) U.S. Cl. CPC B01L 7/52; B01L 3/502761; B01L 9/527; C12Q 1/6844; B01L 2200/16; B01L 2300/1827; B01L 2300/0887

(57) ABSTRACT

A rapid thermal cycling device can include a static microfluidic reaction chamber that can be defined between a layered substrate and a cover that can have an average space therebetween from 4 μm to 150 μm. The layered substrate can include a heating element thermally coupled to the static microfluidic reaction chamber to heat a fluid when present therein. The layered substrate, the cover, or both can include a heat diffusing material thermally coupled to the static microfluidic reaction chamber to diffuse heat out from the fluid while remaining in the static microfluidic reaction chamber.

### BACKGROUND

Nucleic acid amplification is a technique utilized in research, medical diagnostics, and forensic testing. The ability to amplify a small quantity of a sample of a nucleic acid to generate copies of the nucleic acid in the sample can permit research, medical diagnostic, and forensic tests that would not otherwise be practical due to the small quantity of the sample, for example.

### BRIEF DESCRIPTION OF THE DRAWINGS

FIG. 1A schematically illustrates a cross-sectional view of an example rapid thermal cycling device in accordance with the present disclosure;

FIG. 1B schematically illustrates a cross-sectional view of an example rapid thermal cycling device in accordance with the present disclosure;

FIG. 1C schematically illustrates a cross-sectional view of an example rapid thermal cycling device in accordance with the present disclosure;

FIG. 1D schematically illustrates a cross-sectional view of an example rapid thermal cycling device in accordance with the present disclosure;

FIG. 2 schematically illustrates a top view of a microfluidic chip in an example in accordance with the present disclosure;

FIG. 3 schematically illustrates a top view of a microfluidic chip in an example in accordance with the present disclosure;

FIG. 4 schematically illustrates a cross-sectional view of an example rapid thermal cycling system in accordance with the present disclosure; and

FIG. 5 is a flow diagram illustrating an example method of rapidly amplifying a nucleic acid in an example use in accordance with the present disclosure.

### DETAILED DESCRIPTION

Nucleic acid amplification can include denaturing, annealing, and extending nucleic acid chains. Typically, the amplification process utilizes specialized equipment that can be costly and cumbersome, and in some instances a thermal cycle may last over an hour. During denaturing an increased temperature can cause hydrogen bonds between bases in a double stranded nucleic acid sample to break apart resulting in two single strands realized from a formerly double stranded nucleic acid. During annealing, the heated sample can then be cooled, enabling single stranded nucleic acid oligomers, such as primers, to attach to the complimentary nitrogen bases on the single strands of the nucleic acid. During extending of the nucleic acid chain the temperature may be increased, for example, to enable a polymerase enzyme to extend the nucleic acid strand by adding nucleic acid bases. Regardless of the sequence or heating and cooling and the temperatures that are reached during the heating and cooling phases, once the temperature cycling profile is established or approximated, this thermal cycling can be repeated until a desired number of nucleic acid copies, e.g., DNA, are formed, which can for example take from about 10 to about 100 thermal cycles, or 20 to 60 thermal cycles in many instances.

A “thermal cycle” can be based on a range of temperatures used for denaturing, annealing, and extending phases of the amplification, with temperatures raising and lowering for various stages within the cycle. For simplification, a thermal cycle can be defined as a series of temperatures within a range of temperatures defined by the uppermost temperature and a lowermost temperature. The cycle can be determined based on either the uppermost or lowermost temperature of the series of temperatures. For example, if a thermal cycle repeated the following temperatures: 75° C. heated to 95° C. cooled to 45° C. heated to 98° C. cooled to initial temperature for the amplification, then the thermal cycle can be counted based on the phase where the temperatures reaches about 98° C., with the first occurrence defining the last temperature of cycle 1. The same could be said of 45° C., which can also be used as the event that occurs to count or identify the cycle. Regardless of temperatures reached between the two endpoint temperatures, generally the temperature can oscillate between a maximum temperature and a minimum temperature, e.g., general heating and cooling stages of the cycle. However, in another example, the thermal profile can be more continuous, within the timeframes outlined herein, or even without specific set timeframes due to the thermal mass of the device and nucleic acid sample fluid and temperature tolerance for reaction phase. In addition, there are some examples where annealing and extension can be carried out at about the same temperature, which may allow for the device to be used outside of the thermal cycling profiles described generally herein. Still further, in some examples, the amplification or other similar cycling can also be modulated in real time, which often can correspond to modulation of the thermal cycling. Furthermore, there can also be modifications to the thermal cycling during various phases of the processing of the nucleic acid sample fluid, such as modification of an initial phase prior to starting the first amplification cycle. For example, a reverse transcription (RT) process and/or introduction of hot-start PCR enzyme could occur during an initial step. In still further detail, the present disclosure can also provide for isothermal amplification using enzymes supporting processes such as loop-mediated isothermal amplification (LAMP) or recombinase polymerase amplification (RPA).

In accordance with examples of the present disclosure, a rapid thermal cycling device includes a static microfluidic reaction chamber that is defined between a layered substrate and a cover having an average space therebetween from 4 μm to 150 μm. The layered substrate includes a heating element thermally coupled to the static microfluidic reaction chamber to heat a fluid when present therein, and the layered substrate, the cover, or both include a heat diffusing material thermally coupled to the static microfluidic reaction chamber to diffuse heat out from the fluid while remaining in the static microfluidic reaction chamber. In one example, the heating element is thermally coupled to the static microfluidic reaction chamber to heat fluid in the static microfluidic reaction chamber at a rate of 100° C./s to 50,000,000° C./s (e.g., pulses of heat ranging in duration from the order of hundreds of nanoseconds to milliseconds), and the heat diffusion material is thermally coupled to the static microfluidic reaction chamber to diffuse heat from fluid in the static microfluidic reaction chamber at a rate of 1,000° C./s to 10,000,000° C./s. In another example, the cover is a thermal diffusion cover that contributes to heat diffusion from the static microfluidic reaction chamber, and has a thickness from 1 μm to 1,000 μm. In yet another example, the layered substrate is a heat cycling substrate with a thermal resistive layer that defines a portion of a boundary of the static microfluidic reaction chamber and contributes to heat diffusion from the static microfluidic reaction chamber. In a further example, the heating element can be positioned within 200 μm from an interior of the static microfluidic chamber. In another example, the heating element is dimensionally as large or larger in surface area as a static microfluidic reaction chamber interface area where the layered substrate defines the static microfluidic reaction chamber. In yet another example, the heating element includes a resistive heating element, field-effect transistor, p-n junction diode, thin film heater, thermal diode, or a combination thereof, and the heating element includes platinum, aluminum, copper, gold, silver, tantalum, titanium, nickel, tin, zinc, chromium, tungsten silicon nitride, tungsten silicide nitride, tantalum aluminum, nichrome, tantalum nitride, tantalum silicide nitride, chromium silicon oxide, poly-silicon, germanium, oxides, alloys, or a combination thereof. In one example, the heating element is positioned to elevate a temperature of a fluid loaded in the static microfluidic reaction chamber by 55° C. to 95° C. when pulsed on for 0.1 μs to 1 second, and with respect to cooling, the layered substrate, the cover, or both in combination contribute to diffusion of heat from fluid loaded in the static microfluidic chamber in between heating element pulses. In another example, the static microfluidic reaction chamber is included as part of an on-chip, internally controlled, lab-on-a-chip device. In yet another example, the rapid thermal cycling device further includes additional microfluidic chambers, wherein the additional microfluidic chambers are arranged in parallel, in series, or a combination thereof.

In another example, a rapid thermal cycling system is presented. The rapid thermal cycling system includes a rapid thermal cycling device and a detection device. The rapid thermal cycling device includes a static microfluidic reaction chamber that is defined between a layered substrate and a cover that has an average space therebetween from 4 μm to 150 μm. The layered substrate includes a heating element thermally coupled to the static microfluidic reaction chamber to heat a fluid when present therein, and the layered substrate, the cover, or both includes a heat diffusing material thermally coupled to the static microfluidic reaction chamber to diffuse heat out from the fluid while remaining in the static microfluidic reaction chamber. The detection device is operably coupled to the static microfluidic reaction chamber to receive data related to fluid prior to, during, or after heat cycling the fluid within the static microfluidic reaction chamber. In one example, the cover is a thermal diffusion cover that contributes to heat diffusion from the static microfluidic reaction chamber, and has a thickness from 1 μm to 1,000 μm and/or the layered substrate is a heat cycling substrate with a thermal resistive layer defining a portion of a boundary of the static microfluidic reaction chamber and contributing to heat diffusion from the static microfluidic chamber.

In another example, a method of rapidly amplifying a nucleic acid includes loading a nucleic acid-amplifying solution in a static microfluidic reaction chamber of a rapid thermal cycling device, with the rapid thermal cycling device including a static microfluidic reaction chamber that is defined between a layered substrate and a cover having an average space therebetween from 4 μm to 150 μm, and the layered substrate including a heating element thermally coupled to the static microfluidic reaction chamber to heat a fluid when present therein. The layered substrate, the cover, or both includes a heat diffusing material thermally coupled to the static microfluidic reaction chamber to diffuse heat out from the fluid remaining in the static microfluidic reaction chamber. The method also includes thermally cycling the nucleic acid-amplifying solution in the static microfluidic reaction chamber to amplify a nucleic acid of the nucleic acid-amplifying solution, with the thermally cycling including a heating phase with the heating element increasing a temperature of the nucleic acid-amplifying solution and a cooling phase with the layered substrate, the cover, or both reducing the temperature of the nucleic acid-amplifying solution. In one example, the heating phase can include generating heating pulses from the heating element lasting for 0.1 μs to 1 second, and the cooling phase includes time intervals between heating pulses lasting from 1 millisecond to 10 seconds. A full cycle of the thermally cycling includes the heating phase and the cooling phase which can occur within 1 millisecond to 11 seconds, for example. In another example, the amplified nucleic acid can be used to identify the presence of genomic or epigenetic indictors related to an infectious disease, a medical condition, forensics, anti-counterfeiting, host response, genetic mutation, or a combination thereof.

When discussing the rapid thermal cycling device, the rapid thermal cycling system, and/or the method of rapidly amplifying a nucleic acid herein, these discussions can be considered applicable to one another whether or not they are explicitly discussed in the context of that example. Thus, for example, when discussing a static microfluidic reaction chamber related to a rapid thermal cycling device, such disclosure is also relevant to and directly supported in the context of the rapid thermal cycling system, the method of rapidly amplifying a nucleic acid, and vice versa.

Terms used herein will take on the ordinary meaning in the relevant technical field unless specified otherwise. In some instances, there are terms defined more specifically throughout the specification or included at the end of the present specification, and thus, these terms can have a meaning as described herein.

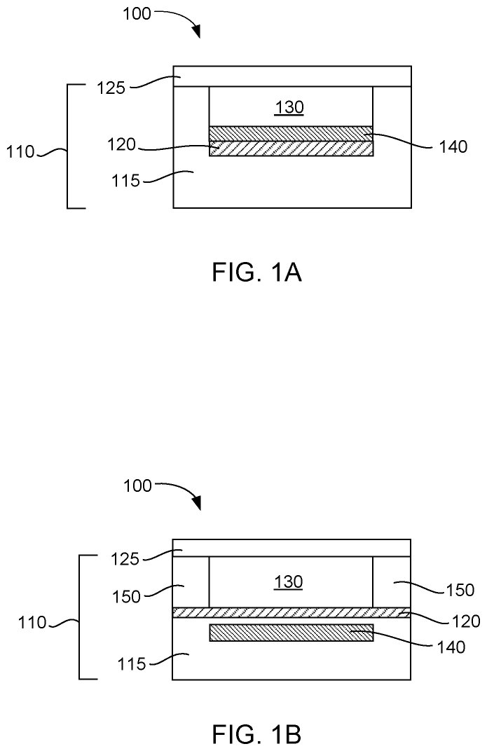

The rapid thermal cycling device 100, as shown by different examples in a cross-sectional view in FIGS. 1A-1D, can include a static microfluidic reaction chamber 130 defined between a layered substrate 110 and a cover 125 or lid. The average space between the layered substrate (from the base thereof, e.g., opposite the cover) and the cover can be from 4 μm to 150 μm. In these examples, the layered substrates can include a support material 115 and a heating element 140 that can be thermally coupled to the static microfluidic reaction chamber so that it can heat a fluid (not shown) when present therein. The heating element can be immediately adjacent to the static microfluidic reaction chamber as shown in FIG. 1A, or the heating element can be carried within the support material and spatially separated (but still thermally coupled) to the static microfluidic reaction chamber 130 as shown in FIG. 1B. In further detail, a thermal resistive layer 120 can be positioned between the support material and the heating element as shown in FIG. 1A, or it can be immediately adjacent to the static microfluidic reaction chamber 130 as shown in FIG. 1B. In FIG. 1B, it is also notable that there is a thickness of support material between the thermal resistive layer and the heating element, which is also another possible arrangement. The thermal resistive layer can likewise be found along the side walls (not shown) for additional heat dissipation in some examples. In FIG. 1B, the side walls 150 are shown as separate layers of the layered substrate, but could be a separate structure, e.g., not part of the layered substrate, or could be provided by a shape or configuration of the cover or lid (see cover 125 in FIG. 1D, for example). FIG. 1A shows the side walls as a unitary structure of the support material.

In some examples, as shown in FIGS. 1C and 1D, the rapid thermal cycling device 100 can include a layered substrate 110 with more layers than that shown and described in FIGS. 1A and 1B. For example, in some examples, there can be multiple heating elements 140a, 140b, electrical contact pads 160, electrical traces (not shown), microfluidic vias and/or channels (not shown), etc., as shown in FIG. 1C. A combination of heating elements can be positioned adjacent to one another or separated from one another, such as by a thermal resistive layer 120. In this example, the two heating elements include resistive heating element 140a and p-n junction diode 140b. The cover 125 in this example also provides the side walls of the chamber, and the layered substrate and the cover define the static microfluidic reaction chamber 130. The height of the static microfluidic reaction chamber can be defined as the space between the layered substrate and the cover, based on surfaces facing one another, for example, e.g., space defined not considering side walls that may be provided by the layered substrate, the cover, or a combination of both. On the other hand, in FIG. 1D, there can be two thermal resistive layers 120 on either side of the heating element 140.

The heating element can be thermally coupled to the static microfluidic reaction chamber to heat fluid in the static microfluidic reaction chamber at a rate of 100° C./s to 50,000,000° C./s (e.g., pulses of heat ranging in duration from the order of hundreds of nanoseconds to milliseconds), from 1,000° C./s to 10,000,000° C./s, or from 5,000° C./s to 1,000,000° C./s, for example.

Regardless of the configuration, the layered substrate (including side walls in some examples), the cover, or both can include a thermal resistive layer, e.g., heat diffusing material layer, thermally coupled to the static microfluidic reaction chamber to diffuse heat out from the fluid while remaining in the static microfluidic reaction chamber. In some examples, the thermal resistive layer can passively diffuse heat out from the fluid while remaining in the static microfluidic reaction chamber. In further detail, the cover and/or the thermal resistive layer can be of a thickness that allows for heat to diffuse from the static microfluidic reaction chamber passively. Thus, the cover and/or the passive thermal resistive layer can dissipate or rapidly remove heat from the static microfluidic reaction chamber. An example thickness for the cover to assist with heat dissipation from the static microfluidic reaction chamber can be from 1 μm to 1,000 μm, from 1 μm to 200 μm, from 5 μm to 1,000 μm, from 5 μm to 200 μm, from 10 μm to 1,000 μm, from 10 μm to 200 μm, from 5 μm to 100 μm, or from 5 μm to 25 μm. An example thickness for the thermal resistive layer to assist with heat dissipation from the static microfluidic reaction chamber can be from 1 μm to 1,000 μm, from 1 μm to 200 μm, from 1 μm to 100 μm, from 1 μm to 25 μm, from 5 μm to 1,000 μm, from 5 μm to 200 μm, from 6 μm to 100 μm, from 5 μm to 25 μm, or from 5 μm to 20 μm. Thus, both materials and thicknesses can be chosen to control heat dissipation rates from the static microfluidic reaction chamber during a cooling cycle, for example. The heat diffusion material, e.g., from the thermal resistive layer, can be thermally coupled to the static microfluidic reaction chamber to diffuse heat from fluid in the static microfluidic reaction chamber at a rate of 1,000° C./s to 10,000,000° C./s, from 2,000° C./s to 5,000,000° C./s, or from 5,000° C./s to 1,000,000° C./s, for example.

In further detail regarding the heating element(s), these components can be thermally coupled to the static microfluidic reaction chamber to heat a fluid when present in the static microfluidic reaction chamber. The heating element 140 can be part of the layered substrate and can be located adjacent to the static microfluidic reaction chamber as shown in FIG. 1A, or the heating element can be recessed in a portion of the layered substrate as shown in FIG. 1B, or both locations may be present as shown in FIG. 1C. Likewise, with reference to the thermal resistive layer 120, this layer(s) can be located adjacent to the static microfluidic reaction chamber as shown in FIG. 1B, or the heating element can be recessed in a portion of the layered substrate as shown in FIG. 1A, or both locations can be present as shown in FIG. 1D.

The types of heating elements that can be used include any on-board heating element that can be included in the rapid thermal cycling device structure. Examples include a resistive heating element, a field-effect transistor, a p-n junction diode, a thin film heater, a thermal diode, or a combination thereof. In one example, the heating element can include a resistive heating element. In another example, the heating element can include a resistive heating element and a p-n junction diode. In yet another example, the heating element can include a resistive heating element and a thermistor.

In some examples, the heating element can include platinum, aluminum, copper, gold, silver, tantalum, titanium, nickel, tin, zinc, chromium, tungsten silicon nitride, tungsten silicide nitride, tantalum aluminum, nichrome, tantalum nitride, tantalum silicide nitride, chromium silicon oxide, poly-silicon, germanium, oxides, alloys, or a combination thereof. In one example, the heating element can include silver. In another example, the heating element can include poly-silicon. In yet another example, the heating element can include tungsten silicon nitride. In a further example, the heating element can include tantalum aluminum.

In one example, the heating element can be dimensionally as large or larger in surface area as one of the surfaces defining the chamber, e.g., a floor surface, a side wall surface, a ceiling surface, etc. If the chamber is not box-like in shape, then the heating element can be as dimensionally large as the largest area of the static microfluidic reaction chamber. Smaller dimensions for the heating element can likewise be used, but rapid thermal cycling can be enhanced when the fluid volume is heated and cooled sufficiently throughout to cycle the full volume of the amplification fluid and/or more evenly distribute heat and cooling profiles for the fluid in the static microfluidic reaction chamber. For example, the heating element can be half of the size in surface area as one of the surfaces defining the chamber, e.g., a floor surface, a side wall surface, a ceiling surface, etc. In another example, the heating element can be three quarters of the size in surface area as one of the surfaces defining the chamber, e.g., a floor surface, a side wall surface, a ceiling surface, etc.

The heating element can be operable to heat a fluid when loaded in the static microfluidic reaction chamber to a temperature ranging from 50° C. to 100° C. The heating temperature can correlate to a temperature activated reaction. For example, a heating temperature for denaturing a double strand of a nucleic acid can range from 80° C. to 100° C. A heating temperature for annealing a complimentary nucleic acid sequence to a single strand of the nucleic acid can range from 50° C. to 65° C. A heating temperature for extending the complimentary nucleic acid sequence can range from 65° C. to 80° C. The denaturing time, annealing time, and extending time can depend on the concentration of reagents and enzyme speeds. In one example, denaturing time, annealing time, and extending time can range from 0.05 seconds to 5 seconds, from 1 second to three seconds, or from 0.05 seconds to 1 second for the various temperature activated reaction. In one example, the heating element can be configured to cycle between temperatures for a specified time period. An example temperature cycle can include heating a fluid in the static microfluidic reaction chamber to a temperature ranging from 80° C. to 100° C. for three seconds, cooling to a temperature ranging from 50° C. to 65° C. for one to two seconds, and heating to a temperature ranging from 65° C. to 80° C. for three seconds.

The heating can be consistent or pulsed. In one example, the heating can be pulsed. Pulsed heating can provide suitable control in heating a fluid in the static microfluidic reaction chamber. In one example, the heating element can be positioned to elevate a temperature of a fluid loaded in the static microfluidic reaction chamber by 20° C. to 50° C. when pulsed on for 0.1 μs to 1 second and the layered substrate, the cover, or both in combination contribute to diffusion of heat from fluid loaded in the static microfluidic chamber in between heating element pulses.

The rapid thermal cycling device can be used to rapidly amplify a nucleic acid on time scale limits imposed by physical and chemical kinetics. For example, in one specific example, a balance of reaction and diffusion kinetics can be present where small devices on the scale described herein can provide a temporal response capable of letting the reaction kinetics of the chemistry of the amplification fluid be a rate-limiting factor, rather than the device properties. In other examples, the device properties may respond more slowly than the reaction kinetics, but even in those instances, the thermal cyclizing can be very fast. For example, either way, the structure of the rapid thermal cycling device can be designed to temperature cycle the fluid therein rapidly, utilizing an internal heating element and a thermal resistive layer for cooling. The rapid thermal cycling device can be used to amplify a nucleic acid within hold times from 0.05 seconds to 10 seconds, from 0.05 seconds to 1 second, from 0.5 seconds to 10 seconds, or from 0.5 seconds to 3 seconds for a denaturing, annealing, and extending phase during the amplification process. The rapid thermal cycling device can be used for polymerase chain reaction, isothermal amplification, loop mediated isothermal amplification (LAMP), recombinase polymerase amplification (RPA), reverse transcription, forward transcription, or a combination thereof. In another example, a continual heat can be applied for isothermal amplification.

The support material of the layered substrate 110 can vary. In one example, the material of the layered substrate and/or side walls can include silicon, silicon dioxide, glass, SUB, bisphenol A novolac epoxy, polymethamethacrylate, polymethacrylate, polystyrene, polycarbonate, or a combination thereof. In another example, the support material and/or side walls can include silicon. In yet another example, the support material and/or side walls can include glass. In yet another example, the support material and/or side walls can include SUB.

The thermal resistive layer of the layered substrate can be any material that rapidly dissipates or removes heat from the static microfluidic reaction chamber for thermal cycling. In one example, the thermal resistive layer can be a passive thermal resistive layer and can rapidly dissipate or remove heat from the static microfluidic reaction chamber without input from an active external device or chemical, such as a cooling device, coolant, a fan or other mechanical cooler, etc. Thus, a passive thermal resistive layer does not rely on an active mechanism to transfer thermal energy from a higher temperature area away to a lower temperature area. In some examples, the heat transfer can occur from a fluid within the static microfluidic reaction chamber to the layered substrate, and more particularly the thermal resistive layer, which can contribute to heat diffusion from the static microfluidic chamber. For clarity, the thermal resistive layer can include multiple layers either adjacent to one another or separated from one another. In one example, the thermal resistive layer can define a portion of a boundary of the static microfluidic reaction chamber. In another example, the thermal resistive layer can be located below the heating element or below a surface of the support material. The heat diffusing material can be located under the static microfluidic reaction chamber, under the heating element, or a combination thereof. The heat diffusing material of the thermal resistive layer can include silicon dioxide, silicon nitride, non-electrically conductive oxides, nitrides, ceramic materials, plastic, diamond, copper, aluminum, silicon, beryllium oxide, boron nitride, or a combination thereof. As mentioned, the heat diffusing layer of the layered substrate can have an average thickness from 1 μm to 1,000 μm, but more typically from 1 μm to 200 μm, from 5 μm to 20 μm, from 10 μm to 50 μm, or from 50 μm to 150 μm.

The cover can be included enclose the static microfluidic reaction chamber. The cover can define an “upper” surface or ceiling of the chamber (as shown in FIGS. 1A and 1B), or can define the upper surface/ceiling and a plurality of side walls of the static microfluidic reaction chamber (as shown in FIG. 1D). It is noted that when referring to upper surfaces, based surfaces, ceilings, side walls, floors, etc., with respect to the static microfluidic reaction chamber, these terms are used for convenience as the chamber can be in any orientation, with the “ceiling” facing sideways or downward, base surface facing downward or sideways, etc. Thus, these terms are to be defined as being relative to one another, and not to define the ultimate orientation of the chamber when loading or in use, for example. With this understanding, the cover can be a removable closure to permit loading of a fluid into that static microfluidic reaction chamber, or the device can be loaded by microfluidic channels with the cover being a more permanently attached structure defined as a “cover” for convenience. In another example, the cover can be affixed to walls or the layered substrate. In other examples, the cover can include an opening configured to permit loading of a fluid into the static microfluidic reaction chamber.

The cover can include a material such as silicon, silicon dioxide, glass, SU8, bisphenol A novolac epoxy, polymethamethacrylate, polymethacrylate, polystyrene, polycarbonate, or a combination thereof. In one example, the cover can include silicon. In yet another example, the cover can include glass. In yet another example, the cover can include SU8. In some examples, the cover can be a thermal diffusion cover that can contribute to heat diffusion from a fluid in the static microfluidic reaction chamber. Thermal diffusion covers can include a heat diffusing material and/or a thermal resistive layer as described above. As mentioned, the cover can have a thickness that can range from 1 μm to 1,000 μm, from 1 μm to 200 μm, from 1 μm to 20 μm, or from 5 μm to 20 μm, for example. A thickness of the cover can contribute to heat retention and heat diffusion from a fluid in the static microfluidic reaction chamber.

A depth of the static microfluidic reaction chamber (vertical height, again without regard to orientation but as a relative measurement between a base of layered substrate and the cover typically facing one another) can contribute to a rate at which a fluid when loaded in the static microfluidic reaction chamber can heat and cool. In one example, the static microfluidic reaction chamber can have a depth that can range from 4 μm to 150 μm. In other examples, the static microfluidic reaction chamber can have a depth that can range from 4 μm to 100 μm, from 10 μm to 90 μm, from 25 μm to 75 μm, or from 10 μm to 20 μm. In one example, the static microfluidic reaction chamber can receive from 1 μL to 10 μL of fluid. In other examples, the static microfluidic reaction chamber can receive from 10 μL to 10 μL of fluid, or from 50 μL to 200 μL of fluid. The quantity of fluid that can be loaded in the static microfluidic reaction chamber can be increased by increasing an area of the interface between the static microfluidic reaction chamber and the heating element or the layered substrate.

The static microfluidic reaction chamber can include a cavity defined by the layered substrate and the cover, with side walls provided by either the layered substrate, the cover, or as a separate standoff structure positioned between the layered substrate and the cover. The static microfluidic reaction chamber can be static in that, denaturing, annealing, and extending of a nucleic acid sample can all occur in the static microfluidic reaction chamber. The nucleic acid sample in this example would not move to subsequent chambers for a cycle of denaturing, annealing, and extending. This can save on processing time, and can be an efficient way to amplify nucleic acids. In some examples, the static microfluidic reaction chamber can be part of a microfluidic chip and a fluid containing a nucleic acid sample can enter and/or exit the static microfluidic reaction chamber before and/or following denaturing, annealing, and extending. That stated, there can be fluid movement from chamber to chamber, but a full cycle of denaturing, annealing, and chain extension can occur in a single chamber, and thus can be considered to be a static microfluidic reaction chamber. In still further detail, the nucleic acid sample fluid or amplification fluid.

Thus, with the static fluid cycling as described herein, in some examples, the rapid thermal cycling device can be integrated in a microfluidic chip. In one example, the microfluidic chip can be an in vitro diagnostic point of care device. The microfluidic chip can include microfluidic channel, microfluidic chamber, circuit component, actuation mechanism, sensing mechanism, temperature controller, detector, or a combination thereof. A microfluidic channel can permit a fluid, such as a nucleic acid amplifying solution, when loaded into the microfluidic chip, to move through the microfluidic chip. When present, a microfluidic channel can have an open or closed arrangement. The microfluidic channels and/or chambers and likewise be vented to allow for fluid movement, for example.

In another example, the microfluidic chip can include additional microfluidic chambers. The additional microfluidic chambers can be arranged in parallel, in series, or a combination thereof. In one example, a microfluidic chip including a microfluidic chamber can permit non-specific amplification of nucleic acids in a fluid sample in the static microfluidic reaction chamber followed by specific amplification of a specific nucleic acid in the microfluidic chamber. For example, the microfluidic chamber for specific amplification can include a primer in lyophilized form that can be reconstituted when a fluid sample enters the microfluidic chamber. In another example, reverse transcription can occur in a microfluidic chamber followed by nucleic acid amplification in the static microfluidic reaction chamber.

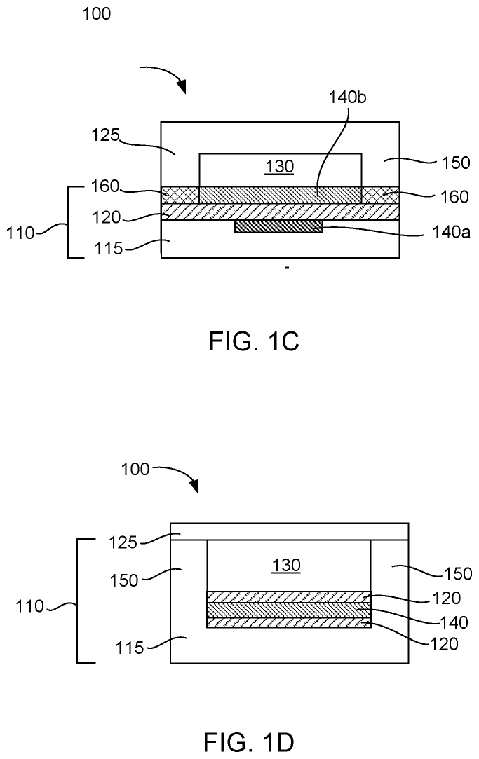

A top view of an example of a microfluidic chip 200 that includes a static microfluidic reaction chamber 210 and a receiving microfluidic chamber 220 is schematically illustrated in FIG. 2. Thus, the microfluidic chip can include a microfluidic port 202 or opening to load a fluid for nucleic acid amplification, the static microfluidic reaction chamber, and a microfluidic channel 204 connecting the static microfluidic reaction chamber to the receiving microfluidic chamber 220. In one example, the static microfluidic reaction chamber is where the nucleic acid amplification can occur, and the receiving microfluidic chamber can be designed for a number purposes, such as a waste receptacle, a detection chamber, a secondary amplification chamber where further amplification occurs, pre-amplification chamber not specifically to concentrate nucleic acids prior to target amplification and detection in the static microfluidic reaction chamber, trapping nucleic acids for subsequent elution into the static microfluidic reaction chamber for reverse transcription and/o amplification, for example. In some examples, a receiving microfluidic chamber can include an air vent that can permit air to escape the receiving microfluidic chamber when a fluid is deposited in the receiving microfluidic chamber.

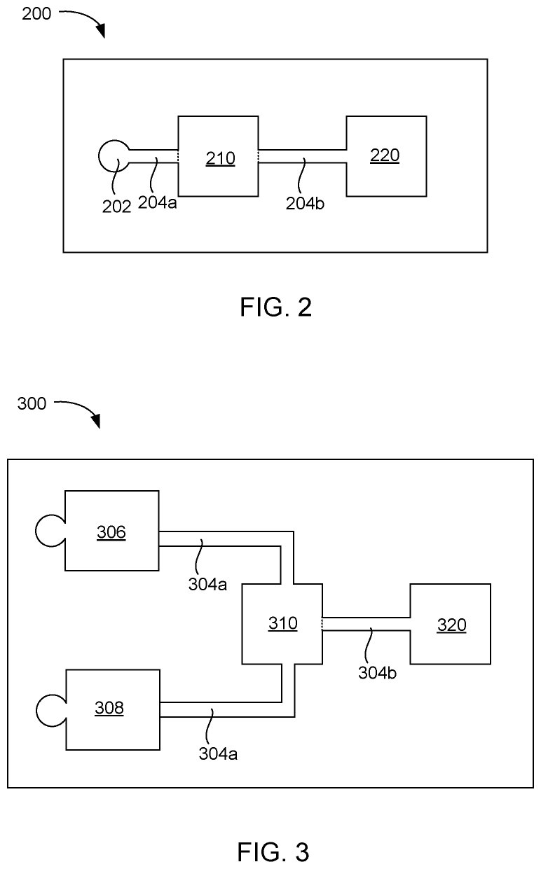

As shown in FIG. 3, in another example, a microfluidic chip 300 can include a first loading microfluidic chamber 306 and a second loading microfluidic chamber 308. The first loading microfluidic chamber can be used to load a fluid sample, for example, and the second loading microfluidic channel can be used to load a reagent to be admixed with the sample in the static microfluidic reaction chamber 310. The two fluids can be introduced into the static microfluidic reaction chamber by microfluidic channels 304a. Microfluidic channel 304b can be used to move the fluid further along into the receiving microfluidic chamber 320 to carry out any of a number of functions. The loading microfluidic chambers are shown in parallel and the static microfluidic reaction chamber and the receiving microfluidic chamber are shown in a series, but the device could be arranged differently depending on the desired function.

Circuit components, when present, can be integrated in the microfluidic chip, e.g., the heating element, or some can be external of the microfluidic chip, e.g., drive circuitry. In one example, the microfluidic chip can include power lines (to bring power if there is no onboard power) and/or control lines (to execute control from an off-chip location, if control does not occur on the chip per se). In other examples, control, actuation, and/or sensing can occur on-board the microfluidic chip. Thus, the circuitry can be more complex with most functions occurring on-board, can be simple with minimal on-board functions, or anything in between that is practical for a microfluidic chip design. Furthermore, circuit components can include a thin film, transistor, vias, substrate connection, interconnect mechanism, or a combination thereof. Circuit components can be arranged serially, in parallel sequence, or a combination thereof. Actuation mechanisms and/or sensing mechanisms can be enabled on a microfluidic chip using complementary metal oxide semiconductor (CMOS) technology, laterally diffused metal oxide semiconductor (LDMOS) technology, bipolar junction transistor (BJT) technology, or a combination thereof. A temperature controller when present can include a heat sensor, a heating element regulator, etc., or a combination thereof. In some examples, a microfluidic chip can further include a detector. The detector can be for detecting fluid, nucleic acid, reagent, a fluorescing chemical compound, or a combination thereof. In some examples, the detector can be a detection device for measuring nucleic acid amplification.

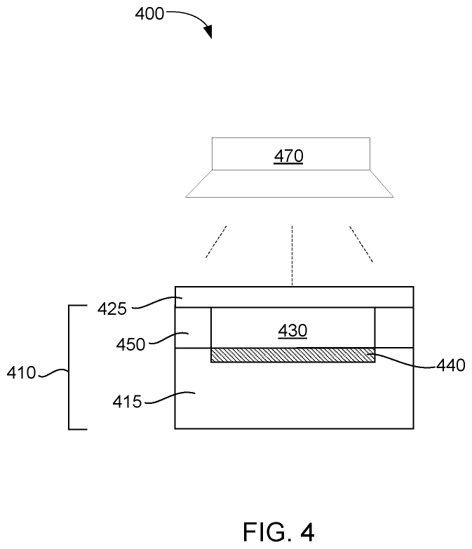

In further detail, a rapid thermal cycling system 400 is shown in FIG. 4 and depicts just a few of the component elements shown and described in FIGS. 1A-1C, but it is understood that any of those structures, or combination of structural elements could be used in the rapid thermal cycling system shown in FIG. 4. In this example, however, the system includes a static microfluidic reaction 430 chamber defined between a layered substrate 410 and a cover 425 having an average space therebetween from 4 μm to 150 μm. The layered substrate can include a support material 415 and a heating element 440 thermally coupled to the static microfluidic reaction chamber to heat a fluid when present therein. The layered substrate and/or side walls 450 thereof can include a thermal resistive layer (not shown, but shown by example in FIGS. 1A-1C). The layered substrate, the cover, or both can include heat diffusing material that can be thermally coupled to the static microfluidic reaction chamber to diffuse heat out from the fluid while remaining in the static microfluidic reaction chamber. The rapid thermal cycling system can further include a detection device 470 operably coupled to the static microfluidic reaction chamber to receive data related to a fluid prior to, during, or after heat cycling of the fluid within the static microfluidic reaction chamber. The detection device is shown schematically, but can include a fluorescent detection system, chemical detection system, chemical luminescence, detection system, optical electrical detection system, etc., or a combination thereof.

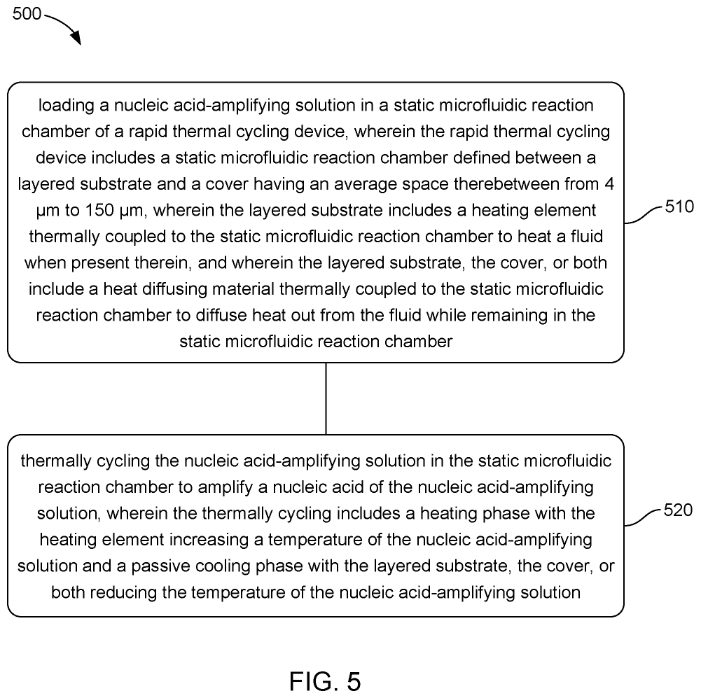

A flow diagram of rapidly amplifying a nucleic acid 500 is shown in FIG. 5. In one example, the method can include loading 510 a nucleic acid-amplifying fluid in a static microfluidic reaction chamber of a rapid thermal cycling device. The rapid thermal cycling device can include a static microfluidic reaction chamber that can be defined between a layered substrate and a cover having an average space therebetween from 4 μm to 150 μm. The layered substrate can include a heating element thermally coupled to the static microfluidic reaction chamber to heat a fluid when present therein. The layered substrate, the cover, or both can include a heat diffusing material thermally coupled to the static microfluidic reaction chamber to diffuse heat out from the fluid while remaining in the static microfluidic reaction chamber. The method can further include thermally cycling 520 the nucleic acid-amplifying fluid in the static microfluidic reaction chamber to amplify a nucleic acid of the nucleic acid-amplifying fluid. The thermally cycling can include a heating phase with the heating element increasing a temperature of the nucleic acid-amplifying fluid and a cooling phase with the layered substrate, the cover, or both reducing the temperature of the nucleic acid-amplifying fluid. In some examples, the method can further include pre-cycling and/or post-cycling hold times between thermal cycles.

The nucleic acid amplifying fluid can include a nucleic acid sample; reagent components such as nucleic acid primers, any of a number of polymerase enzymes, e.g., Taq polymerase enzyme, nucleotides; buffering components; water; organic co-solvents; minerals; other liquid vehicle components, etc.

The heating phase and cooling phase can follow rapid thermal cycling kinetics. In one example, the heating phase can include generating heating pulses from the heating element lasting from 0.1 μs to 1 second, from 0.1 μs to 100 milliseconds, from 1 μs to 50 milliseconds, from 10 μs to 50 milliseconds, from 100 μs to 10 milliseconds, or from 1 millisecond to 1 second, for example. Heating pulses can be applied as a single pulse, or a series of pulses. The (passive, in one example) cooling phase can include time intervals between two heating phases lasting from 1 millisecond to 10 seconds, from 1 millisecond to 5 seconds, from 1 millisecond to 1 second, from 1 millisecond to 0.5 seconds, from 10 milliseconds to 10 seconds, from 10 milliseconds to 5 seconds, from 10 milliseconds to 1 second, from 10 milliseconds to 0.5 seconds, from 100 milliseconds to 10 seconds, from 100 milliseconds to 5 seconds, from 100 milliseconds to 1 second, from 100 milliseconds to 0.5 seconds, etc. A full cycle of the thermally cycling which includes the heating phase and the cooling phase can occur at from about 1 millisecond to about 11 seconds, about 10 milliseconds to about 10 seconds, or about 100 milliseconds to about 5 seconds. In some examples, the cooling phase can exclude active cooling, and can thus, be considered passive cooling. For cooling passively within the millisecond timeframes described herein, a lid or cover with a thickness of about 5 μm to about 25 μm, e.g., about 10 μm, can dissipated heat out of the reaction chamber within a range of milliseconds, e.g., 1 millisecond to 500 milliseconds. This, in combination with the thermal resistive layer also acting to dissipate heat from the reaction chamber, rapid cooling even faster than 1 millisecond can occur.

The method can be used to amplify a nucleic acid sample that can include mRNA, RNA, DNA, or a combination thereof. In one example, the amplified nucleic acid can be used to diagnose a medical condition and/or detect the presence of a disease. In some examples, the method can be used to diagnose gonococcal infections, neisserial infections, trachomatis infections, Mycobacterium tuberculosis, Neisseria gonorrhea, Chlamydia trachomatis, malaria, HIV, AIDS, or a combination thereof. Furthermore, the devices, systems, and methods described herein can also be used to amplify anything else with DNA or RNA using PCR amplification, for example, e.g., forensics, pathogen detection beyond human disease, anti-counterfeiting, etc. Furthermore, the capability of this type of nucleic acid amplification can be applied in the area of nucleic acid amplification testing (NAAT), which may include pathogen detection for infectious diseases, host response, or other epigenetic impact. In cancer testing, for example, the technique can be used to detect genetic mutations such as BRAF or KRAF. In essence, the devices, systems, and methods here can be applied to applications where amplification is use, such as in the case of concentrating nucleic acids prior to sequencing, e.g., DNA sequencing. In some examples, the diagnosis, detection, or some other similar function utilizing amplification as described herein can occur within 20 minutes of loading a nucleic acid sample into the rapid thermal cycling device. In some examples, the diagnosis can occur within 30 seconds to 20 minutes, 1 minute to 20 minutes, 1 minutes to 10 minutes, 1 minute to 5 minutes, 2 minutes to 20 minutes, 2 minutes to 10 minutes, 2 minutes to 5 minutes, etc., of loading a nucleic acid sample into the rapid thermal cycling device.

It is noted that, as used in this specification and the appended claims, the singular forms “a,” “an,” and “the” include plural referents unless the content clearly dictates otherwise.

As used herein, a plurality of items, structural elements, compositional elements, and/or materials may be presented in a common list for convenience. However, these lists should be construed as though the individual member of the list is identified as a separate and unique member. Thus, no individual member of such list should be construed as a de facto equivalent of any other member of the same list based on the presentation in a common group without indications to the contrary.

Concentrations, dimensions, amounts, and other numerical data may be presented herein in a range format. A range format is used merely for convenience and brevity and should be interpreted flexibly to include the numerical values explicitly recited as the limits of the range, as well as to include all the individual numerical values or sub-ranges encompassed within that range as the individual numerical value and/or sub-range is explicitly recited. For example, a weight ratio range of about 1 wt % to about 20 wt % should be interpreted to include the explicitly recited limits of 1 wt % and 20 wt % and to include individual weights such as about 2 wt %, about 11 wt %, about 14 wt %, and sub-ranges such as about 10 wt % to about 20 wt %, about 5 wt % to about 15 wt %, etc.

### EXAMPLES

The following illustrates examples of the present disclosure. However, the following is illustrative of the application of the principles of the present disclosure. Numerous modifications and alternative compositions, methods, and systems may be devised without departing from the disclosure. The appended claims are intended to cover such modifications and arrangements.

### Example 1—Preparation of a Rapid Thermal Cycling Device

A 0.1 μm thick tungsten silicon nitride (WSiN) resistive heating element having a square resistivity of 400 Ohm/m² is centrally adhered to a 5 μm thick silicon dioxide oxide layer on a 675 μm thick silicon substrate. 21 μm tall side wall standoff structures are built up on the silicon substrate immediately adjacent to a periphery of the heating element. The heating element is positioned such that the heating element is thermally coupled to a static microfluidic reaction chamber defined by the heating element as a floor surface, and silicon side walls. A 100 μm silicon dioxide cover is removably positioned over the static microfluidic reaction chamber. An average space between the heating element and the cover is 100 μm. The silicon dioxide layer on the substrate and the cover are configured to act as a heat diffusing material to passively diffuse heat out from a fluid when present in the static microfluidic reaction chamber.

### Example 2—Preparation of a Rapid Thermal Cycling Device

A 0.1 μm thick TaAl resistive heating element is centrally adhered to a 10 μm thick SU8 layer on a 750 μm thick SU8 substrate. 17 μm tall side wall standoff structures are built up on the SU8 substrate immediately adjacent to a periphery of the heating element. The heating element is positioned such that the heating element is thermally coupled to a static microfluidic reaction chamber defined by the heating element as a floor surface, and silicon side walls. A 20 μm SU8 cover is removably positioned over the static microfluidic reaction chamber. An average space between the heating element and the cover is 17 μm. The SU8 layer on the substrate and the cover are configured to act as a heat diffusing material to passively diffuse heat out from a fluid when present in the static microfluidic reaction chamber.

### Example 3—Preparation of a Rapid Thermal Cycling Device

A 9 μm thick lead resistive heating element is centrally adhered to a 15 μm thick glass layer on a 200 μm thick glass substrate. 4 μm tall side wall standoff structures are built up on the glass substrate immediately adjacent to a periphery of the heating element. The heating element is positioned such that the heating element is thermally coupled to a static microfluidic reaction chamber defined by the heating element as a floor surface, and glass side walls. A 20 μm glass cover is removably positioned over the static microfluidic reaction chamber. An average space between the heating element and the cover is 4 μm. The glass layer on the substrate and the cover are configured to act as a heat diffusing material to passively diffuse heat out from a fluid when present in the static microfluidic reaction chamber.

### Example 4—Rapid Thermal Cycling System

A rapid thermal cycling device created in Example 1 is packaged with an optical detection device operably coupled to the static microfluidic reaction chamber to receive data related to the nucleic acid amplifying fluid prior to, during, or after thermal cycling within the static microfluidic reaction chamber.

### Example 5—Rapid Thermal Cycling

A nucleic acid-amplifying fluid including the components in Table 1 is loaded in a static microfluidic reaction chamber of a rapid thermal cycling device prepared in accordance with Example 1.

TABLE 1
Nucleic Acid-Amplifying Solution

| Component | Amount |
|---|---|
| PCR Grade Water | 7.6 μL |
| Bovine Serum Albumin | 2.5 mg/μL |
| 25 mM MgCl₂ Stock | 2.4 μL |
| β Globin Primer Mix | 2.0 μL |
| Sybr Green | 2.0 μL |
| 0.357 U/μL Enzyme Soln. | 2.0 μL |
| Kit Master Mix | 2.0 μL |
| β Globin DNA | 2.0 μL |
| KAPA2G Fast DNA Polymerase* | 5 U/μL |

*KAPA2G Fast DNA Polymerase is commercially available from former Kapa Biosystems, Inc. (Massachusetts), now part of Roche Company

The heating element was used to heat the nucleic acid-amplifying solution to 94° C. for 100 milliseconds to denature double stranded nucleic acid. The heating element cycled off and the nucleic acid-amplifying solution is allowed to cool to 56° C. over a period of 400 milliseconds where the temperature is held (by lower heat level) for 200 milliseconds for annealing of the denatured nucleic acid with primer, for example. The temperature of the heating element is increased to 70° C. and held for 400 milliseconds for chain extension. This cycle is repeated 45 times. The nucleic acid sample amplified is two times per cycle. The rapid thermal cycling device permitted the nucleic acid and other reagents of the nucleic acid amplifying fluid to be loaded in the static microfluidic reaction chamber wherein the amplification occurred by thermal cycling. A reaction rate per cycle where denaturing, annealing, and extending phases occurred took about 1 second per cycle, with 3.5×10¹³ copies of the nucleic acid prepared from one starting copy in a total time of 45 seconds.

While the present technology has been described with reference to certain examples, it will be appreciated that various modifications, changes, omissions, and substitutions can be made without departing from the spirit of the disclosure. It is intended, therefore, that the disclosure be limited only by the scope of the following claims.

What is claimed is:

1. A rapid thermal cycling device, comprising a static microfluidic reaction chamber defined between a layered substrate and a cover having an average space therebetween from 4 μm to 150 μm, wherein the layered substrate includes a heating element thermally coupled to the static microfluidic reaction chamber to heat a fluid when present therein, and wherein the layered substrate, the cover, or both include a heat diffusing material thermally coupled to the static microfluidic reaction chamber to diffuse heat out from the fluid while remaining in the static microfluidic reaction chamber.

2. The rapid thermal cycling device of claim 1, wherein the heating element is thermally coupled the static microfluidic reaction chamber to heat fluid in the static microfluidic reaction chamber at a rate of 100° C./s to 50,000,000° C./s and the heat diffusion material is thermally coupled to the static microfluidic reaction chamber to diffuse heat from fluid in the static microfluidic reaction chamber at a rate of 1,000° C./s to 1,000,000° C./s.

3. The rapid thermal cycling device of claim 1, wherein the cover is a thermal diffusion cover that contributes to heat diffusion from the static microfluidic chamber, and has a thickness from 1 μm to 1,000 μm.

4. The rapid thermal cycling device of claim 1, wherein the layered substrate is a heat cycling substrate with a thermal resistive layer defining a portion of a boundary of the static microfluidic reaction chamber and contribute to heat diffusion from the static microfluidic chamber.

5. The rapid thermal cycling device of claim 1, wherein the heating element is positioned within 200 μm from an interior of the static microfluidic chamber.

6. The rapid thermal cycling device of claim 1, wherein the heating element is dimensionally as large or larger in surface area as a static microfluidic reaction chamber interface area where the layered substrate defines the static microfluidic reaction chamber.

7. The rapid thermal cycling device of claim 1, wherein the heating element includes a resistive heating element, a field-effect transistor, a p-n junction diode, thin film heater, thermal diode, or a combination thereof, and wherein the heating element includes platinum, aluminum, copper, gold, silver, tantalum, titanium, nickel, tin, zinc, chromium, tungsten silicon nitride, tungsten silicide nitride, tantalum aluminum, nichrome, tantalum nitride, tantalum silicide nitride, chromium silicon oxide, poly-silicon, germanium, oxides, alloys, or a combination thereof.

8. The rapid thermal cycling device of claim 1, wherein the heating element is positioned to elevate a temperature of fluid loaded in the static microfluidic reaction chamber by 20° C. to 50° C. when pulsed on for 0.1 μs to 1 second, and the layered substrate, the cover, or both in combination contribute to diffusion of heat from fluid loaded in the static microfluidic chamber in between heating element pulses.

9. The rapid thermal cycling device of claim 1, wherein the static microfluidic reaction chamber is included as part of an on-chip, internally controlled, lab-on-a-chip device.

10. The rapid thermal cycling device of claim 1, further comprising additional fluid chambers, wherein the additional fluid chambers are arranged in parallel, in series, or a combination thereof.

11. A rapid thermal cycling system, comprising:
a static microfluidic reaction chamber defined between a layered substrate and a cover having an average space therebetween from 4 μm to 150 μm, wherein the layered substrate includes a heating element thermally coupled to the static microfluidic reaction chamber to heat a fluid when present therein, and wherein the layered substrate, the cover, or both include a heat diffusing material thermally coupled to the static microfluidic reaction chamber to diffuse heat out from the fluid while remaining in the static microfluidic reaction chamber; and
a detection device operably coupled to the static microfluidic reaction chamber to receive data related to fluid prior to, during, or after heat cycling the fluid within the static microfluidic reaction chamber.

12. The rapid thermal cycling system of claim 11, wherein:
the cover is a thermal diffusion cover that contributes to heat diffusion from the static microfluidic chamber, and has a thickness from 1 μm to 1,000 μm;
the layered substrate is a heat cycling substrate with a thermal resistive layer defining a portion of a boundary of the static microfluidic reaction chamber and contribute to heat diffusion from the static microfluidic chamber; or
both.

13. A method of rapidly amplifying a nucleic acid, comprising:
loading a nucleic acid-amplifying solution in a static microfluidic reaction chamber of a rapid thermal cycling device, wherein the rapid thermal cycling device includes a static microfluidic reaction chamber defined between a layered substrate and a cover having an average space therebetween from 4 μm to 150 μm, wherein the layered substrate includes a heating element thermally coupled to the static microfluidic reaction chamber to heat a fluid when present therein, and wherein the layered substrate, the cover, or both include a heat diffusing material thermally coupled to the static microfluidic reaction chamber to diffuse heat out from the fluid while remaining in the static microfluidic reaction chamber; and
thermally cycling the nucleic acid-amplifying solution in the static microfluidic reaction chamber to amplify a nucleic acid of the nucleic acid-amplifying solution, wherein the thermally cycling includes a heating phase with the heating element increasing a temperature of the nucleic acid-amplifying solution and a cooling phase with the layered substrate, the cover, or both reducing the temperature of the nucleic acid-amplifying solution.

14. The method of claim 13, wherein the heating phase includes generating heating pulses from the heating element lasting for 0.1 μs to 1 second, and the cooling phase includes time intervals between heating phases lasting from 1 millisecond to 10 seconds, and wherein a full cycle of the thermally cycling which includes the heating phase and the cooling phase occurs from about 1 millisecond to about 11 seconds.

15. The method of claim 13, wherein the amplified nucleic acid is used to identify the presence of genomic or epigenetic indictors related to an infectious disease, a medical condition, forensics, anti-counterfeiting, host response, genetic mutation, or a combination thereof.
